(19) United States
(12) Patent Application Publication
(10) Pub. No.: US 2007/0173903 A1
(43) Pub. Date: Jul. 26, 2007

(54) Medical device for restoration of neurological function impaired by peripheral neuropathy

(75) Inventors: Andy Ofer Goren, Newport Beach, CA (US); Yehuda G. Goren, Scotts Valley, CA (US); Peter Novak, Jamaica Plain, MA (US); Elliott J. Stein, Morristown, NJ (US); Christopher Chi-Chuen Chen, Wallace, CA (US); Amy Morningstar, Scotts Valley, CA (US)

Correspondence Address: THELEN REID BROWN RAYSMAN & STEINER LLP, P. O. BOX 640640, SAN JOSE, CA, 95164-0640, US

(73) Assignee: BioQ, Inc., Newport Beach, CA (US)

(21) Appl. No.: 11/512,739

(22) Filed: Aug. 29, 2006

Related U.S. Application Data
(60) Provisional application No. 60/712,976, filed on Aug. 30, 2005.
(60) Provisional application No. 60/831,035, filed on Jul. 13, 2006.

(51) Int. Cl. A61N 1/00; A63B 21/00; A61B 5/103

(52) U.S. Cl. 607/49; 600/595; 607/77; 607/144

(57) ABSTRACT

A device for treating a patient using sensory substitution includes a wearable article in which are disposed one or more sensors for detecting the phase of the gait cycle of the patient, a controller for receiving signals from the sensors indicative of the phase of the gait, and one more stimulators for stimulating the patient based on signals from the controller that are issued in response to the sensor signals.

### CROSS-REFERENCE TO RELATED APPLICATIONS

This application claims the benefit of U.S. provisional patent application No. 60/712,976, filed on Aug. 30, 2005, entitled “Medical Device for Treatment of Balance and Gait Disorders Using Sensory Substitution,” and U.S. provisional patent application No. 60/831,035, filed on Jul. 13, 2006, entitled “Therapeutic Device for Prevention of Ulcerations Using Sensory Substitution,” both of which are incorporated herein by reference.

### BACKGROUND OF THE INVENTION

1. Field of the Invention

The invention relates to the treatment of peripheral neuropathy disorders.

2. Description of Related Art

A major problem facing diabetic patients suffering from peripheral neuropathy as well as the general aging population is the increased risk of falls during walking. During human gait, transmission of cutaneous feedback from the feet is essential for maintaining normal gait and balance. Non-nociceptive cutaneous feedback from the feet is normally transduced via mechanoreceptors at the sole and transmitted via the afferent nerve fibers to the central nervous system.

It is well documented in the medical literature that diabetic peripheral neuropathy results in functional loss of nerve fibers which is usually irreversible and has no medical treatment currently available. The loss of nerve fibers is characterized by severe sensory deficit of vibrational and tactile perception.

Another problem facing diabetic patients suffering from peripheral neuropathy is the increased risk of developing foot ulcerations. The decrease in cutaneous feedback from the feet of diabetic patients suffering from peripheral neuropathy and the associated gait impairment results in the development of abnormal planar pressure during human gait. Abnormal planar pressure results in abnormal repetitive stress to the feet and thus increases the risk of developing foot ulcerations.

Various devices have been proposed to attempt to improve abnormal cutaneous feedback from the feet in patients with neuropathy. One approach stimulates the patients feet with “noise”—that is, random sub-threshold mechanical or electrical stimulation in order to reduce the threshold of cutaneous mechanoreceptors. A shortcoming of this approach is that the stimulation intensity needs to adjusted individually for each patient and the long term effectiveness of the treatment remains unclear. In another approach the patient's feet are stimulated using supra-threshold vibratory mechanical stimulation in order to overcome the increased stimulus threshold of the cutaneous mechanoreceptors. Shortcomings of this approach include the potential for nerve damage due to repetitive supra-threshold vibratory mechanical stimulation, the lack of effectiveness of the device in subjects with severe peripheral neuropathy, and the practical means of energizing a device embedded in a subject's shoe.

There therefore exist a need for a system that overcomes the limitations of previous approaches by providing a wearable, low cost, self contained device that stimulates a subject's skin area less affected by peripheral neuropathy in accordance with the phase of the gait cycle in order to treat balance and gait disorders and prevent problems associated with abnormal planar pressure resulting in abnormal repetitive stress to the feet and increasing the risk of developing foot ulcerations.

### SUMMARY OF THE INVENTION

The current invention makes use of the phenomenon of sensory substitution. Sensory substitution is a known neurological phenomenon whereby a subject with a failed or degraded mode of perception learns that an input signal from a different modality of perception on the subject's body is used to complement the failed or degraded perception. In accordance with one embodiment of the invention, there is provided a device for providing neural sensory substitution. The device includes one or more sensors configured to generate acceleration signals in response to a human gait during the human gait cycle, a controller configured to determine phases of the human gait cycle using the acceleration signals and to issue control signals in accordance with the determined gait phases, and one or more stimulators configured to stimulate a wearer of the device in response to the control signals.

In accordance with another embodiment of the invention, there is provided a device for treating a gait disorder of a patient. The device includes an article that is wearable by patient, one or more sensors coupled to the article and configured to generate acceleration signals in response to the gait of the patient, a controller configured to determine phases of the gait of the patient using the acceleration signals and to issue control signals in accordance with the determined phases, and one or more stimulators configured to stimulate the patient in response to the control signals.

In accordance with yet another embodiment of the invention, there is provided a device for reducing the risk and/or preventing the formation of foot ulcerations in diabetic patients. The device includes an article that is wearable by patient, one or more sensors coupled to the article and configured to generate acceleration signals in response to the gait of the patient, a controller configured to determine phases of the gait of the patient using the acceleration signals and to issue control signals in accordance with the determined phases, and one or more stimulators configured to stimulate the patient in response to the control signals.

### BRIEF DESCRIPTION OF THE SEVERAL VIEWS OF THE DRAWINGS

Many advantages of the present invention will be apparent to those skilled in the art with a reading of this specification in conjunction with the attached drawings, wherein like reference numerals are applied to like elements, and wherein:

FIG. 1 is perspective view of a device 10 worn on the leg of a patient and utilizing sensory substitution;

FIG. 2 is a cross-sectional view of the device 10 of FIG. 1;

FIG. 3 is a schematic view of components of a system comprising device 10 of FIG. 1; and

FIG. 4 is schematic view of a device in the form of a footwear 25 utilizing sensory substitution.

### DETAILED DESCRIPTION OF THE INVENTION

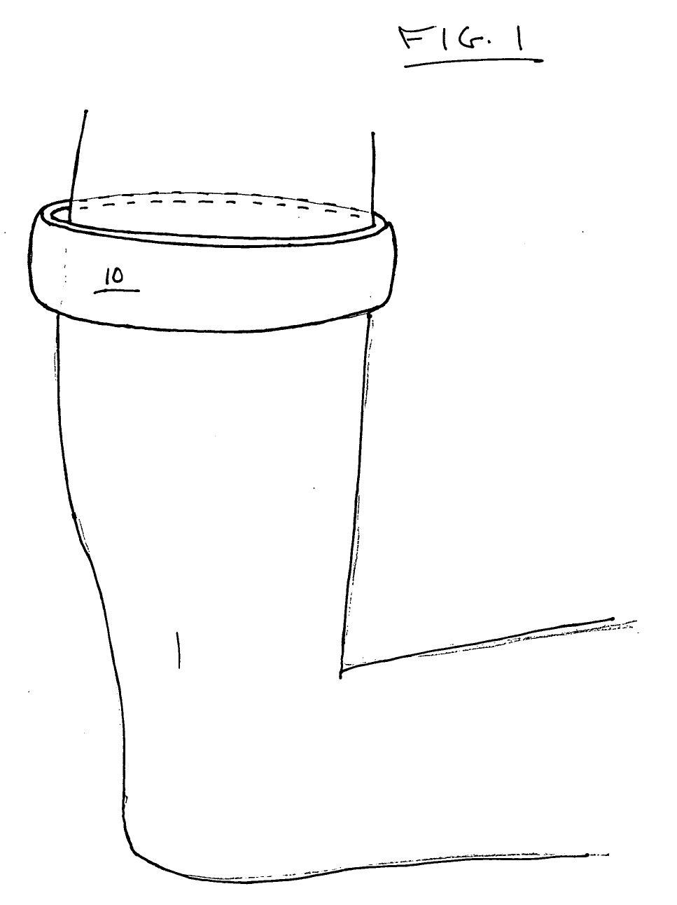

FIG. 1 is a perspective view of a therapeutic device in the form of a cuff 10 worn on the leg of a patient for treating balance or gait disorders as well as reduction of risk of ulcerations. The cuff or similar worn article may be in the form of a conforming, comfortable elastic band of suitable durability and compatibility with the skin of the wearer. While the preferred location for wearing the cuff is the leg, other places are also contemplated, such as the arm or wrist (bracelet), neck, sole of the foot, ankle, and so forth.

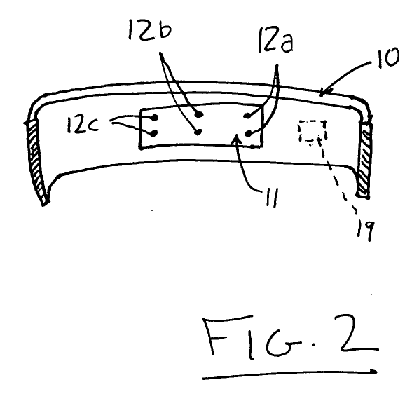

FIG. 2 is a cross-sectional cut-away view of cuff 10, showing a contact pad 11 on an interior surface of the cuff intended to make contact with the skin of the patient when the cuff is worn. Contact pad 11 has a set of six stimulators grouped in pairs 12a, 12b and 12c that are disposed respectively in anterior, central and posterior portions of the contact pad. It will be appreciated that the number, grouping and location of the stimulators are not critical. More or less than six may be used, and these may or may not be grouped in pairs, and may or may not be disposed symmetrical in the contact pad. The particular arrangement of stimulators should be selected such that optimum stimulation effect of the patient is achieved thereby. One example of a selectable arrangement of stimulators is a geometrical pattern that mimics the location of the contact points of the human foot with the ground during the human gait cycle.

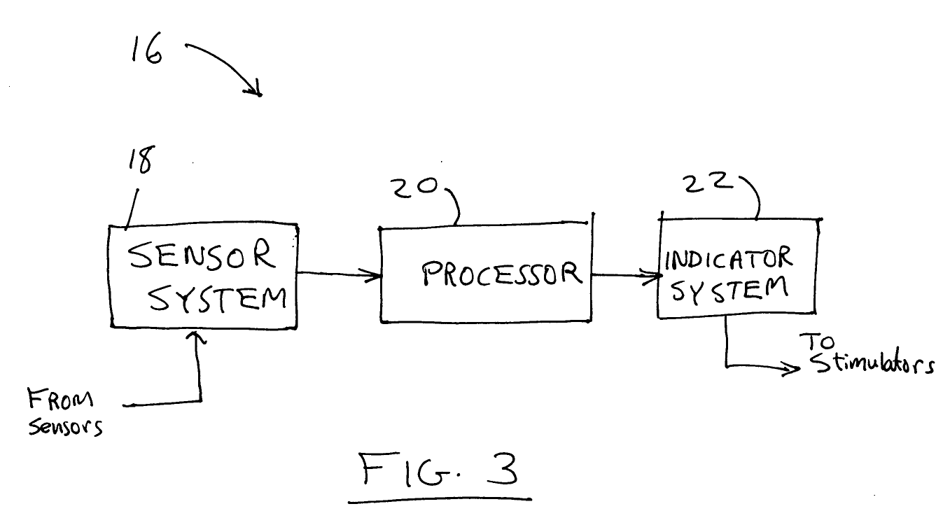

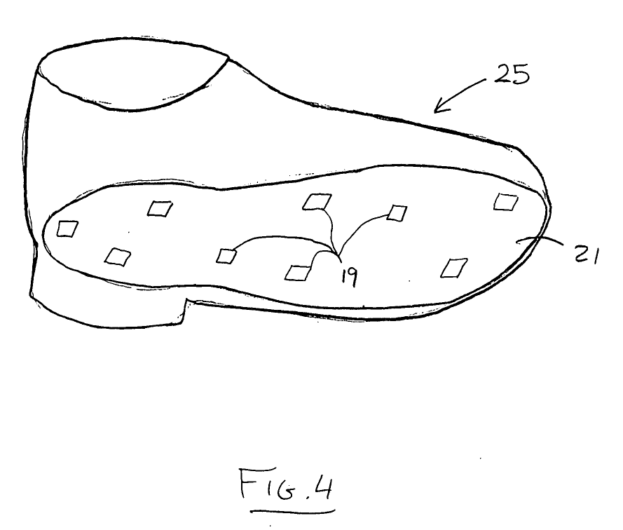

FIG. 3 is a schematic diagram of a therapeutic system 16 included with cuff 10. A sensor system 18 provides input signals to a controller or processor 20, which in turn activates an indicator system 22 accordingly. The processor 20 may be “hard-wired” to perform as desired, or it may be programmable such that its functions can be tailored to the particular patient's needs and the device fitted accordingly. In the preferred embodiment, the indicator system includes the stimulators 12a, 12b and 12c. The sensor system 18 is designed to provide information to the processor 20 to thereby enable the processor to distinguish and/or predict various phases of the gait cycle. The gait cycle is the time between any two identical walking events during human walking. Each gait cycle is divided into a stance and swing period. The stance period constitutes 62 percent of the gait cycle and is composed of 5 phases: initial contact, loading response, midstance, terminal stance, and preswing. The swing period constitutes 38 percent of the gait cycle and is composed of 3 phases: initial swing, midswing, and terminal swing. Sensor system 18 includes one or more acceleration-measuring sensors (that is, accelerometers) 19 housed in cuff 10 (FIG. 2). Alternatively, sensors 19 may be housed in a separate device or cuff (not shown) worn by the patient and communicating with the cuff 20 wirelessly or with a wire. The sensors 19 of sensor system 18 are designed to pick up accelerations during the human gate cycle, caused for instance by the impact of parts of the foot, such as the heel or toes, against the ground, and/or accelerations of the foot during its swing between ground contacts, and/or accelerations induced by lifting of the foot from the ground. The information from the sensors 19, including the direction and magnitude of the accelerations and their point of occurrence for instance as coinciding with ground impact, is forwarded to the processor 20, which translates the information into a representation of the phase of the gait cycle. Alternatively, sensor system 18 can be in the form of one or more pressure-sensors 21 embedded in a specially-fitted portion 23 of a shoe 25 worn by the patient, as shown in FIG. 4. While portion 23 is shown to correspond to the insole of the shoe 25, other footwear components or portions of the shoe, in lieu of or in addition to the insole, can be so outfitted. In addition, the system 16 itself can be housed in a shoe or similarly-wearable device, dispensing with the need to provide cuff 10. Another possibility is in the form of a sock for example. The information from the sensors 21 is forwarded to the processor 20, which translates the information into a representation of the patient's gait cycle. Communication between the sensors 21 and processor 20 would preferably take place wirelessly, and suitable power sources, transmitters, and receivers (not shown) for effecting this, disposed in the shoe 23 and the cuff 10, would be provided as necessary. It may also be advantageous, depending on the application, to use sensors in the form of a gyroscope, or a piezoelectric device.

The information from sensor system 18 as translated by processor 20 into the representation of the patient's gait cycle, is used to effect selective activation of the indicators 22, and in particular, stimulators 12a, 12b and 12c, to thereby provide the patient with feedback regarding his/her position and possible magnitude in the gate cycle. The stimulators 12a, 12b and 12c are mapped to correspond to different regions of the foot, preferably but not necessarily in a correspondence with the portion of the foot that would normally be most activated during the particular phase of the gait cycle. Specifically, anterior stimulators 12a correspond to the front of the foot or the toes, and are activated when this portion of the foot is for example determined by the processor 20 to be in contact with the ground, particularly during the push-off phase of the gait cycle. Central stimulators 12b are activated when the foot is flat against the ground, for example during mid-stance. Posterior stimulators 12c are activated during heel strike or initial contact. Of course, combinations of stimulators 12a-12c can be activated at various times during the gait cycle. Further, the activation can be suitably timed to account for impulse travel times, reaction times, and so forth in order to provide optimum effect. Further, as stated above, while three sets of stimulators are described, more or fewer sets, grouped differently and consisting of more or fewer than two can also be used. In addition, indicators other than or in addition to the stimulators can be used, including auditory and/or visual indicators. Also as mentioned above, a suitable power supply would be provided in the cuff to drive system 16, and can include a rechargeable battery pack (not shown). Power can also be obtained from a non-battery source, or from an electromechanical source which converts kinetic energy into electrical energy.

The system 16 is designed to provide feedback to the patient to help the patient maintain balance or otherwise improve his/her gait. It is also intended to provide feedback to the patient in order to address the problem of foot ulcerations due to abnormal planar pressure. In addition, since the system uses sensory substitution by providing feedback to a different location from that from which information about the gait is normally derived physiologically, patients with a markedly reduced feeling, for example in their feet, can still benefit since they would receive information, through stimulators 12a, 12b and 12c, at the location of the cuff, which can be tailored to the patient's needs and is not limited to the leg location shown in FIG. 1.

Depending on the type of acceleration sensors 19 used, their location within cuff 10 may or may not be critical, based on the direction of motion of the patient's leg. Further, while described in terms of correcting gait disorders, it will be appreciated that balance or stance disorders can also be addressed. Sensors/acceleration detectors that can pick up patient motion in a lateral direction would be useful in such systems, particularly in a direction that is perpendicular or transverse to the gait direction, for example in the direction of“swaying.”

An example of an accelerometer that can be used to detect balance and gait disorders in humans is a low-g accelerometer such as the ADXL203™ by Analog Devices. The ADXL203™ can detect acceleration components in up to 2 independent perpendicular axes. Each acceleration component can detect an acceleration in the range of +/−1.7 g. The ADXL203™ has a very high sensitivity of 1000 mV/g which is critical in the sway detection as well as a very low energy consumption of up to 2.1 mW power at 3 V battery source. Finally, the ADXL203™ is extremely light and compact size—that is, as small as 5 mm×5 mm×2 mm, and weighing less that 0.5 gram.

The stimulators 12a, 12b and 12c are selected to provide mechanical supra-threshold neuronal stimulation to the skin mechanoreceptors of the patient. Alternatively or in addition, the stimulators 12a, 12b and 12c can be selected to provide transcutaneous electrical stimulation to the skin mechanoreceptors. To optimize the effect of the stimulators, an adjustment mechanism may be provided to adjust the intensity of the stimulations they provide. Adjustment may also be desired so as to provide the patient with phase or magnitude information relating to the cycle. Further, intensity adjustment may be effected automatically by the controller or processor 20. The controller may be configured to activate and deactivate one or more of the stimulators in a temporal pattern to provide the wearer with phase information relating to the gait cycle. The phase information can also be indicated by using a pattern of stimulation frequencies.

The above are exemplary modes of carrying out the invention and are not intended to be limiting. It will be apparent to those of ordinary skill in the art that modifications thereto can be made without departure from the spirit and scope of the invention as set forth in the following claims.

1. A device for providing neural sensory substitution, comprising:
one or more sensors configured to generate acceleration signals in response to a phase and/or a phase change of a human gait cycle;
a controller configured to determine phases of said human gait cycle using said acceleration signals and to issue control signals in accordance with said determined phases; and
one or more stimulators configured to stimulate a wearer of the device in response to said control signals.

2. The device of claim 1, wherein at least one stimulator provides mechanical supra-threshold neuronal stimulation to skin mechanoreceptors of the wearer.

3. The device of claim 1, wherein at least one stimulator provides transcutaneous electrical stimulation to skin mechanoreceptors of the wearer.

4. The device of claim 1, wherein at least one stimulator provides auditory or visual stimulation to the wearer.

5. The device of claim 1, wherein the acceleration signals are generated based on lifting of a foot of the wearer from ground during the human gait cycle.

6. The device of claim 1, wherein the acceleration signals are generated based on impact of a foot of the wearer with ground during the human gait cycle.

7. The device of claim 1, wherein the acceleration signals are generated in response to acceleration in a direction that is transverse to a direction of the wearer's gait.

8. The device of claim 1, wherein the controller selectively activates the sensors based on prediction of phases of the gait of the wearer.

9. The device of claim 1, further including a housing in which the controller, at least one of the one or more sensors, and at least one of the one or more stimulators are housed.

10. The device of claim 1, wherein the device is in the form of a wearable cuff.

11. The device of claim 1, wherein the device is in the form a wearable footwear component.

12. The device of claim 1, further comprising:
a first wearable component in which is disposed at least one sensor; and
a second wearable component in which is disposed at least one stimulator,
wherein the controller is disposed in one of the first or second wearable components and communicates wirelessly or via wired means with at least one sensor and/or at least one stimulator.

13. The device of claim 1, wherein at least one of the one or more sensors is a gyroscope.

14. The device of claim 1, wherein at least one of the one or more sensors is an accelerometer.

15. The device of claim 1, wherein at least one of the one or more sensors is a pieozoelectric sensor.

16. The device of claim 1, further comprising a rechargeable power source.

17. The device of claim 1, further comprising an electromechancial power source.

18. The device of claim 1, wherein the one or more stimulators are arranged in a geometrical pattern mimicking contact points of a human foot with ground during a human gait cycle.

19. The device of claim 1, said device configured for use to treat gait and balance disorders in peripheral neuropathy patients.

20. The device of claim 1, said device configured for use to prevent and/or reduce falls in peripheral neuropathy patients.

21. The device of claim 1, said device configured for use to reduce abnormal foot planar pressure during walking in peripheral neuropathy patients.

22. The device of claim 1, said device configured for use to prevent and/or reduce the formation of foot ulcerations in peripheral neuropathy patients.

23. The device of claim 1, wherein at least one of the one or more stimulators has adjustable stimulation strength.

24. The device of claim 23, wherein adjustment of the stimulator strength includes activating and/or deactivating a stimulator and is based on phase information of the gait of the wearer.

25. The device of claim 23, wherein adjustment of the stimulator strength includes activating and/or deactivating a stimulator to provide a temporal stimulation pattern based on phase information of the gait of the wearer.

26. The device of claim 23, wherein adjustment of the stimulator strength includes activating and/or deactivating a stimulator to provide stimulation in a pattern of frequencies based on phase information of the gait of the wearer.

27. A device for restoring neurological function impaired by peripheral neuropathy in a patient comprising:
an article that is wearable by patient;
one or more sensors coupled to the article and configured to generate acceleration signals in response to a phase and/or a phase change of a gait cycle of the patient;
a controller configured to determine phases of the gait of the patient using said acceleration signals and to issue control signals in accordance with said determined phases; and
one or more stimulators configured to stimulate the patient in response to said control signals.

28. The device of claim 27, wherein at least one stimulator provides mechanical supra-threshold neuronal stimulation to skin mechanoreceptors of the wearer.

29. The device of claim 27, wherein at least one stimulator provides transcutaneous electrical stimulation to skin mechanoreceptors of the wearer.

30. The device of claim 27, wherein at least one stimulator provides auditory or visual stimulation to the wearer.

31. The device of claim 27, wherein the acceleration signals are generated based on lifting of a foot of the wearer from ground during the human gait cycle.

32. The device of claim 27, wherein the acceleration signals are generated based on impact of a foot of the wearer with ground during the human gait cycle.

33. The device of claim 27, wherein the acceleration signals are generated in response to acceleration in a direction that is transverse to a direction of the wearer's gait.

34. The device of claim 27, wherein the controller selectively activates the sensors based on prediction of phases of the gait of the wearer.

35. The device of claim 27, further including a housing in which the controller, at least one of the one or more sensors, and at least one of the one or more stimulators are housed.

36. The device of claim 27, wherein the controller is programmable.

37. The device of claim 27, wherein the device is in the form of a wearable cuff.

38. The device of claim 27, wherein the device is in the form a wearable footwear component.

39. The device of claim 27, further comprising:
a first wearable component in which is disposed at least one sensor; and
a second wearable component in which is disposed at least one stimulator,
wherein the controller is disposed in one of the first or second wearable components and communicates wirelessly or via wired means with at least one sensor and/or at least one stimulator.

40. The device of claim 27, wherein at least one of the one or more sensors is a gyroscope.

41. The device of claim 27, wherein at least one of the one or more sensors is an accelerometer.

42. The device of claim 27, wherein at least one of the one or more sensors is a pieozoelectric sensor.

43. The device of claim 27, further comprising a rechargeable power source.

44. The device of claim 27, further comprising an electromechancial power source.

45. The device of claim 27, wherein the one or more stimulators are arranged in a geometrical pattern mimicking contact points of a human foot with ground during a human gait cycle.

46. The device of claim 27, said device configured for use to treat gait and balance disorders in peripheral neuropathy patients.

47. The device of claim 27, said device configured for use to prevent and/or reduce falls in peripheral neuropathy patients.

48. The device of claim 27, said device configured for use to reduce abnormal foot planar pressure during walking in peripheral neuropathy patients.

49. The device of claim 27, said device configured for use to prevent and/or reduce the formation of foot ulcerations in peripheral neuropathy patients.

50. The device of claim 27, wherein at least one of the one or more stimulators has adjustable stimulation strength.

51. The device of claim 27, wherein said controller adjusts the one or more stimulator's stimulation strength to provide the patient with phase information of the human gait cycle and/or sway information.

52. The device of claim 27, wherein said controller activates and deactivates the one or more stimulator's stimulation in a temporal pattern to provide the patient with phase information of the human gait cycle and/or sway information.

53. The device of claim 27 wherein said controller activates and deactivates the one or more stimulator's stimulation in a pattern of frequencies to provide patient with phase information of the human gait cycle and/or sway information.

54. The device of claim 1, wherein the controller is programmable.
